(19) United States
(12) Patent Application Publication
(10) Pub. No.: US 2017/0020800 A1
(43) Pub. Date: Jan. 26, 2017

(54) ORAL COMPOSITIONS

(71) Applicant: 3M INNOVATIVE PROPERTIES COMPANY, St. Paul, MN (US)

(72) Inventors: Yizhong Wang, Woodbury, MN (US); Joel D. Oxman, Minneapolis, MN (US)

(21) Appl. No.: 15/302,275

(22) PCT Filed: Apr. 14, 2015
(86) PCT No.: PCT/US2015/025689

§ 371 Date: Oct. 6, 2016

Related U.S. Application Data
(60) Provisional application No. 61/979,036, filed on Apr. 14, 2014.

(51) Int. Cl. A61K 8/81; A61K 8/22; A61Q 11/00; A61K 8/34

(52) U.S. Cl. CPC A61K 8/8152; A61K 8/34; A61K 8/345; A61K 8/22; A61Q 11/00

(57) ABSTRACT

An oral composition is described. The oral composition can include a solvent comprising water and a cosolvent chosen from lower alkyl alcohols, acetone and a combination thereof, and an acidic copolymer comprising of carboxylic acid monomeric units. The acidic copolymers can be dissolved in the oral composition and the oral composition can form a film on a surface when contacted with an aqueous solution.

### FIELD

The present disclosure generally relates to oral compositions.

### BACKGROUND

The color of teeth can be improved by a number of methods and approaches, such as whitening, cleaning by scaling and polishing, and placing crowns and veneers. Many application techniques, such as brushing, bleaching strips, bleaching pen, bleaching gel and laser bleaching, have been used for tooth whitening.

Existing tooth whitening products include tooth whitening gels and tooth whitening strips. Typically, tooth whitening gels are aqueous/glycerol or propylene glycol based gels. These tooth whitening gels require trays to hold the gels to avoid gel being washed away by saliva or lip movement. Tooth whitening strips are generally for home use with low peroxide concentration and loading.

### SUMMARY

Some aspects of the present disclosure provide an oral composition. The oral composition can include a solvent comprising water and a cosolvent chosen from lower alkyl alcohols, acetone and a combination thereof, and an acidic copolymer comprising of carboxylic acid monomeric units. The oral composition can include from about 5 to about 30 wt % of water, from about 30 to about 65 wt % of cosolvent, from about 10 to about 40 wt % of acidic copolymer, and the wt % of each component is based on the total weight of the composition. The acidic copolymers can be dissolved in the oral composition and the oral composition can form a film on a surface when contacted with an aqueous solution.

Some aspects of the present disclosure provide a method of delivering an oral composition to a dental structure. The method can include providing the oral composition of the present disclosure; applying the oral composition to the dental structure; and contacting the oral composition with an aqueous solution, thereby forming a polymeric film on the dental structure.

Other features and aspects of the present disclosure will become apparent by consideration of the detailed description.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

The present disclosure generally relates to oral compositions. Particularly, for example, the oral compositions of the present disclosure can provide an easily removable and a non sticky, smooth feeling film on dental structures when the oral compositions are contacted with the dental structures and then set by aqueous solution or optional air dry for some instances.

As used herein, dental structures include, but are not limited to, dental tissues and dental articles.

As used herein, dental tissues include, but are not limited to hard and soft dental tissues. Hard and soft oral tissues include, but not limited to, teeth, dental arch, and the surrounding tissues and support structures including gingiva and the hard palate.

As used herein, dental articles include, but are not limited to an article that can be attached (e.g., bonded) to dental tissues (e.g., a tooth structure). Examples of dental articles include, but are not limited to, replacements, inlays, onlays, veneers, full and partial crowns, bridges, implants, implant abutments, copings, dentures, posts, bridge frameworks and other bridge structures, abutments, orthodontic appliances and devices including, but not limited to archwires, buccal tubes, brackets and bands, and prostheses (e.g., partial or full dentures).

As used herein, an aqueous solution includes, but is not limited to water, saliva, artificial saliva or combinations thereof.

In some embodiments, the oral composition of the present disclosure can include a solvent and an acidic copolymer.

In some embodiments, the solvent can include water and a cosolvent. In some other embodiments, the oral composition comprises from about 5 to about 30 wt % of water. In other embodiments, the oral composition comprises from about 10 to about 28 wt % of water.

The cosolvent can be chosen from lower alkyl alcohols and acetone. As referred to herein, the lower alkyl alcohols can include low carbon number (e.g. C₁-C₅) alcohols. Examples of lower alkyl alcohols as used herein include, but are not limited to, ethanol, isopropanol, propylene glycol, glycerin and ethylene glycol based ester alcohols. In some embodiments, the cosolvent can be ethanol. In some other embodiments, the oral composition comprises from about 30 to about 65 wt % of cosolvent. In some other embodiments, the oral composition comprises from about 35 to about 60 wt % of cosolvent.

In some embodiments, the solvent can further include at least one additional component chosen from isopropanol, propylene glycol, glycerin, low molecular weight polyethylene glycol, ethylene glycol based ester alcohols, and combinations thereof. In other embodiments, the cosolvent can comprise propylene glycol.

The acidic copolymer can include acidic functional groups, including for example, carboxylic acid groups, phosphoric acid groups, sulfuric acid groups, sulfonic acid groups, and combinations of any two or more thereof.

In some embodiments, the acidic copolymer can be dissolved in the oral composition.

In some embodiments, the oral composition can be a single homogenous phase, such as for example, a single homogeneous liquid phase which is flowable.

The acidic copolymers can be used as film formers. When the film is formed, it can, for example, provide an anchoring structure to dental tissues and promote such tissues to enhance uptake active agents.

The acidic copolymers of the present invention can include an acidic acrylic copolymer. Such copolymers comprise monomeric units selected from the group consisting of acrylic acid, methacrylic acid, maleic acid, maleic anhydride, itaconic acid, itaconic acid anhydride and combinations thereof. The acidic copolymers of the present invention can include an acrylic copolymer of a first monomeric unit selected from the group consisting acidic monomer and combinations thereof and a second monomeric unit selected from the group consisting of acrylates, acrylamides, vinyl acetates and combinations thereof. The first monomeric units can include methacrylic acid or acrylic acid. Second monomeric units can include acrylates, acrylamides, vinyl acetates and combinations thereof. In one embodiment, the second monomeric unit comprises one or more acrylates. Acrylates can include isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, and 1,4-butanediol acrylate. Acrylates and methacrylate monomers can include the acrylates and methacrylate of: 1,4-butanediol, 1,6-hexanediol, tetraethylene glycol, tripropylene glycol, and ethoxylated bisphenol-A, trimethylol propane, glyceryl propoxy bisphenol-A, and pentaerythritol. Acrylates can further include methacrylates such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate. 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate and trifluoroethyl methacrylate.

The acidic copolymer can have acidic acrylate monomeric units, acidic methacrylate monomeric units, or a combination thereof.

In some embodiments, the acidic copolymer can include, but is not limited to, an acidic acrylic copolymer of a monomeric unit selected from the group consisting of acrylic acid, methacrylic acid and combinations thereof.

In some embodiments, the acidic copolymer can include a copolymer of methacrylic acid and methyl methacrylate. In some other embodiments, the acidic copolymer can include Eudragit® S100 (marketed by Evonic Industries AG, Damstadt, Germany), Eudragit® L100 (marketed by Evonic Industries AG, Damstadt, Germany), Eudragit® L100-55 (marketed by Evonic Industries AG, Damstadt, Germany), AC210 (marketed by The Lubrizol Corporation, Wickliffe, Ohio, USA), or combinations thereof.

In some embodiments, the molecular weight of the acidic copolymer can be about 5,000 Da, about 10,000 Da, about 25,000 Da, about 50,000 Da, about 75,000 Da, about 100,000 Da, about 150,000 Da, about 200,000 Da, about 250,000 Da, about 300,000 Da, about 350,000 Da, about 375,000 Da, about 400,000 Da, about 450,000 Da, about 500,000 Da, or can be a range between and including any two of these values, such as for example from about 5,000 Da to about 500,000 Da.

In some embodiments, the oral composition can comprise from about 10 to about 40 wt % of the acidic copolymers. In other embodiments, the oral composition can comprise from about 15 to about 35 wt % of the acidic copolymers. When the wt % of the acidic copolymer is too high, the oral composition may be difficult to handle and mix. When the wt % of the acidic copolymer is too low, it may be difficult to form a film with the oral composition.

In some embodiments, the oral composition can form a film on a surface when contacted with an aqueous solution. In some embodiments, the aqueous solution includes water, saliva, artificial saliva, or combinations thereof. In other embodiments, the aqueous solution comprises at least about 90 wt %, at least about 95 wt %, or at least about 99 wt % water. In some other embodiments, the oral composition of the present disclosure can form a film in less than about 30 seconds after the oral composition is contacted with the aqueous solution. In other embodiments, the oral composition of the present disclosure can form the film in less than about 5 seconds after the oral composition is contacted with the aqueous solution.

When the oral composition of the present disclosure contacts water, the water miscible solvents can diffuse into water and water can also diffuse into the oral composition. As a result, the molecular interaction among the copolymer chains can increase dramatically and form a soft, elastic and slippery film. In some embodiments, the oral composition can be no longer flowable after the oral composition is contacted with the aqueous solution or air-dried.

The oral composition of the present disclosure can provide a coated film. In some embodiments, at least 90% of the film can be removed after brushing the surface for less than 10 strokes. In some other embodiments, at least 90% of the film can be removed after brushing the surface for less than 5 strokes.

Various methods can be employed to apply the oral composition. In some embodiments, the oral composition can be applied from the compositions container or dispenser such as a bottle, syringe, or tube. In some embodiments, a dental brush, microfiber, foam or sponge applicator or cotton swab is used to rub the surface of a dental structure and leave a thin coating on the surface. In some other embodiments, a tray applicator or a dental tray filled with the oral composition can be used. The oral composition can cover the surface of the dental structure and leave a layer of film on the surface. In other embodiments, the oral composition can be directly painted onto the surface of the dental structure with a brush tip attached to a syringe. The oral composition can be set into a film on the dental structure and its attachments within 30 seconds by water or saliva. In some embodiments, the oral composition can be applied to teeth under moist conditions (e.g., applied to the teeth of healthy patient with typical levels of moisture in the oral cavity without drying the teeth or surfaces thereof prior to application of the oral composition).

In some embodiments, the oral composition of the present disclosure can include active agents. In other embodiments, the active agents can include, but are not limited to whitening agents.

In various embodiments, the oral compositions of the present disclosure can include a whitening agent. As further discussed below, a “whitening agent” is a material which is effective to effect whitening of a tooth surface to which it is applied. In various embodiments, the oral compositions of the present disclosure can include a peroxide whitening agent, comprising a peroxide compound. As referred to herein, a “peroxide compound” is an oxidizing compound comprising a bivalent oxygen-oxygen group. Peroxide compounds can include, but are not limited to, peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and, alkaline earth metals can include, but are not limited to, lithium peroxide, potassium peroxide, sodium peroxide, magnesium peroxide, calcium peroxide, barium peroxide, and mixtures thereof. Organic peroxy compounds can include, but are not limited to, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts can include, but are not limited to, organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound can include, but are not limited to, hydrogen peroxide, urea peroxide adduct (often referred to simply as “urea peroxide” or “UHP”), sodium percarbonate and mixtures thereof. In one embodiment, the peroxide compounds can include hydrogen peroxide. In one embodiment, the peroxide compound can consist essentially of hydrogen peroxide.

The oral compositions of the present disclosure can include one or more non-peroxide whitening agent, in addition to or instead of, peroxide compounds. Whitening agents among those useful herein can include non-peroxy compounds, such as chlorine dioxide, chlorites and hypochlorites. Chlorites and hypochlorites can include, but are not limited to, those of alkali and alkaline earth metals such as lithium, potassium, sodium, magnesium, calcium and barium. Non-peroxide whitening agents can also include, but are not limited to, colorants, such as titanium dioxide and hydroxyapatite.

The following embodiments are intended to be illustrative of the present disclosure and not limiting.

### Embodiments

Embodiment 1 is an oral composition, comprising:

a solvent comprising water and a cosolvent chosen from lower alkyl alcohols, acetone and a combination thereof; and

an acidic copolymer comprising of carboxylic acid monomeric units;

wherein the oral composition comprises from about 5 to about 30 wt % of water, from about 30 to about 65 wt % of cosolvent, from about 10 to about 40 wt % of acidic copolymer, and the wt % of each component is based on the total weight of the composition;

wherein the acidic copolymer is dissolved in the oral composition;

wherein the oral composition is capable of forming a film on a surface when contacted with an aqueous solution;

and wherein at least 90% of the film is removed after brushing the surface for less than 10 strokes.

Embodiment 2 is the oral composition of embodiment 1, wherein the oral composition is capable of forming the film in less than about 30 seconds after the oral composition is contacted with aqueous solution.
Embodiment 3 is the oral composition of any preceding embodiment, wherein the oral composition is capable of forming the film in less than about 5 seconds after the oral composition is contacted with aqueous solution.
Embodiment 4 is the oral composition of any preceding embodiment, wherein at least 90% of the film is removed after brushing the surface for less than 5 strokes.
Embodiment 5 is the oral composition of any preceding embodiment, wherein the cosolvent is ethanol.
Embodiment 6 is the oral composition of any preceding embodiment, wherein the solvent further comprises at least one additional component chosen from isopropanol, propylene glycol, glycerin, low molecular weight polyethylene glycol, ethylene glycol based ester alcohols, and combinations thereof.
Embodiment 7 is the oral composition of any preceding embodiment, wherein the cosolvent comprises propylene glycol.
Embodiment 8 is the oral composition of any preceding embodiment, wherein the oral composition comprises from about 10 to about 28 wt % of water.
Embodiment 9 is the oral composition of any preceding embodiment, wherein the oral composition comprises from about 35 to about 60 wt % of the cosolvent.
Embodiment 10 is the oral composition of any preceding embodiment, wherein the molecular weight of the acidic copolymer is from about 5,000 Da to about 500,000 Da.
Embodiment 11 is the oral composition of any preceding embodiment, wherein the oral composition comprises from about 15 to about 35 wt % of the acidic copolymer.
Embodiment 12 is the oral composition of any preceding embodiment, wherein the acidic copolymer is an acidic copolymer comprising acidic acrylate monomeric units, acidic methacrylate monomeric units, or a combination thereof.
Embodiment 13 is the oral composition of any preceding embodiment, wherein the acidic copolymer is chosen from Eudragit S100, Eudragit L100, Eudragit L100-55, AC210 and combinations thereof.
Embodiment 14 is the oral composition of any preceding embodiment, further comprising an active agent.
Embodiment 15 is the oral composition of embodiment 14, wherein the active agent is a whitening agent.
Embodiment 16 is the oral composition of embodiment 15, wherein the whitening agent is a peroxide whitening agent.
Embodiment 17 is the oral composition of embodiment 16, wherein the peroxide whitening agent is chosen from hydrogen peroxide, peroxides of alkali and alkaline earth metals, organic peroxy compounds, peroxy acids, peroxyacetic acid, urea hydrogen peroxide adduct, pharmaceutically-acceptable salts thereof, and mixtures thereof.
Embodiment 18 is the method of delivering an oral composition to a dental structure comprising:

providing the oral composition of embodiment 1;

applying the oral composition to the dental structure; and

contacting the oral composition with an aqueous solution, thereby forming a polymeric film on the dental structure.

### Examples

Objects and advantages of this invention are further illustrated by the following examples, but the particular materials and amounts thereof recited in these examples, as well as other conditions and details, should not be construed to unduly limit this invention.

The materials used to prepare examples of the invention (E) as well as comparative examples (CE) are outlined below.

### Materials

Oral (Coating) Composition Components

| Component | Description | Source |
|---|---|---|
| EUDRAGIT L100-55 | Acidic anionic copolymer based | Evonik Industries (Darmstadt, |
| | on methacrylic acid and ethyl | Germany) |
| | acrylate | |
| Hydrogen peroxide | 30% H₂O₂ aqueous solution | Avanto (Center Valley, PA) |
| | (w/w) | |
| Urea hydrogen peroxide adduct | 97% | Alfa Aesar (Ward Hill, MA) |
| Ethanol | 190 proof, USP | Columbus Chemical Industries |
| | | (Columbus, WI) |
| DI water | Deionized water | 3M (St. Paul, MN) |
| Propylene glycol | USP grade | EMD (Billerica, MA) |
| Glycerol | USP grade | Sigma-Aldrich (St. Louis, MO) |
| AEROSIL 200 Pharma | silica | Evonik Industries (Parsippany, |
| | | NJ) |

### General Preparation of Oral (Coating) Compositions

Polymer solutions were prepared by first weighing a specified amount of solvent(s) (e.g., ethanol, DI water, propylene glycol, and/or glycerol into a 250 mL glass jar equipped with a cap. Specified amounts of the copolymer component and other components (solvents and/or peroxide) were then added to the jar. The jar was sealed and placed on a Wheaton Culture Roller for 2-3 days (˜30 rpm) until the copolymer component was completely dissolved in the solvent(s) as to provide a copolymer solution. Additional components such as aqueous H₂O₂, silica, and urea hydrogen peroxide adduct were added to the copolymer solution using two×2 minute cycles in a speed mixer (SpeedMixer DAC150.1 FVZ available from FlacTek, Inc., Landrum, S.C.) set at 3000 rpm. Aqueous H₂O₂ dissolved in the copolymer solution. Silica was used to form a gel for rheology control. The components used in each coating composition as well as the amount (in grams) are shown in the examples and tables which follow.

### Test Methods

### Coating Evaluation—Set and Adhesion

Bovine teeth were coated with the indicated coating compositions by painting the tooth surface with a tiny dental brush and the resultant coated teeth were dipped in water for 5 seconds. Setting of the coatings was evaluated by finger touch. The liquid coatings set into soft and slightly elastic films. Adhesion of the films was tested by rinsing the coated teeth in water for 30 seconds. No coatings were rinsed off, indicating reasonable adhesion.

### Coating Removability Testing Method—Tooth Brush Testing

Bovine teeth were potted in a poly(methyl)methacrylate resin and then polished with 320 grit sandpaper to expose the enamel surface. The exposed enamel surfaces were wiped with a paper towel to remove excess water. The exposed enamel surfaces of the teeth were coated with the coating composition, dipped in water for 5 seconds to set the liquid coating into solid film, then stored in glass vial under moist conditions in an oven set to 37° C. for 40 minutes. A tooth brush machine (available from Foth Production Solutions, LLC, Green Bay Wis.), was used to test the removability of the coating from the enamel surface. A water and toothpaste mixture (5 mL of a 1:1 mixture of Crest cavity protection toothpaste:water) was used as the brushing media. The brushing stroke is defined as brushing the surface back and forth one time. After the indicated number of brushing strokes, the tooth surfaces were evaluated to determine whether the coating was removed from the enamel surface. Three bovine teeth were used for every coating sample.

### Whitening Evaluation

Bovine teeth were coated with indicated coating compositions, dipped in water for 5 seconds to form a soft wrap around teeth, then stored in a glass vial in an oven set to 37° C. for the indicated time. After the whitening process, the bovine teeth were rinsed with water. The wrap was then easily brushed away under moist conditions. After rinsing with water, the color change of teeth was evaluated by comparing the photos before and after whitening. The teeth were clearly whitened under different conditions. The tooth color change was also evaluated by comparison color/shade change before and after whitening using a VITA classic shade guide from Vident (Brea, Calif.).

TABLE 1
Coating Composition Examples E1-E6 and Comparative
Examples CE1-CE2

| Component | E1 | E2 | E3 | E4 | E5 | E6 | CE1 | CE2 |
|---|---|---|---|---|---|---|---|---|
| EUDRAGIT | 18 | 18 | 35 | 30 | 20 | 18 | 35 | 35 |
| L100-55 | | | | | | | | |
| Ethanol | 52 | 47 | 65 | 60 | — | — | 45 | 45 |
| DI water | — | — | — | 10 | — | — | 20 | 20 |
| Propylene | — | — | — | — | 70 | 62 | — | — |
| glycol | | | | | | | | |
| 30% H₂O₂ | 25 | 30 | 66 | — | — | 20 | 33 | 66 |
| aqueous | | | | | | | | |
| solution (w/w) | | | | | | | | |
| Glycerol | 5 | 5 | — | — | — | — | — | — |
| Polymer | Yes | Yes | Yes | Yes | Yes | Yes | No | No |
| solution | | | | | | | | |
| Formed? | | | | | | | | |

TABLE 2
Coating Composition Examples E7-E14

| Component | E7 | E8 | E9 | E10 | E11 | E12 |
|---|---|---|---|---|---|---|
| From Exp 4 | 10 | 10 | 10 | — | 10 | 10 |
| From Exp 5 | — | — | — | 10 | — | — |
| From Exp 6 | — | — | — | — | — | — |
| Urea hydrogen | 8 | — | 5 | 5 | — | 5 |
| peroxide adduct | | | | | | |
| AEROSIL 200 | 0.7 | 1.2 | 1.2 | 0.9 | 1.2 | 1.2 |
| Pharma | | | | | | |
| 30% H₂O₂ aqueous | — | 5 | 5 | 3 | 5 | 5 |
| solution (w/w) | | | | | | |
| Set in water by 5 | Yes | Yes | Yes | Yes | Yes | Yes |
| seconds | | | | | | |
| Adhesion without | Good | Good | Good | Good | Good | Good |
| coating rinse off | | | | | | |
| Tooth color | Yes | Yes | Yes | Yes | Yes | Yes |
| changed after | | | | | | |
| whitening? | | | | | | |

TABLE 3
Whitening Efficacy in Comparison to VITA Classic Shade Guide

| Coating | | |
|---|---|---|
| Example | Whitening Conditions | Color/Shade Change |
| E7 | 37° C. × 30 minutes | From A4 to A1 |
| E10 | 37° C. × 30 minutes | From A4 to B2 |

TABLE 4
Coating Removability Test Results

| Coating Example | Strokes to Brush Away Coating |
|---|---|
| E7 | 10 |
| E10 | 10 |

1. An oral composition, comprising:
a solvent comprising water and a cosolvent chosen from lower alkyl alcohols, acetone and a combination thereof; and
an acidic copolymer comprising of carboxylic acid monomeric units;
wherein the oral composition comprises from about 5 to about 30 wt % of water, from about 30 to about 65 wt % of cosolvent, from about 10 to about 40 wt % of acidic copolymer, and the wt % of each component is based on the total weight of the composition;
wherein the acidic copolymer is dissolved in the oral composition;
wherein the oral composition is capable of forming a film on a surface when contacted with an aqueous solution;
and wherein at least 90% of the film is removed after brushing the surface for less than 10 strokes.

2. The oral composition of claim 1, wherein the oral composition is capable of forming the film in less than about 30 seconds after the oral composition is contacted with aqueous solution.

3. The oral composition of claim 1, wherein the oral composition is capable of forming the film in less than about 5 seconds after the oral composition is contacted with aqueous solution.

4. The oral composition of claim 1, wherein the at least 90% of the film is removed after brushing the surface for less than 5 strokes.

5. The oral composition of claim 1, wherein the cosolvent is ethanol.

6. The oral composition of claim 1, wherein the solvent further comprises at least one additional component chosen from isopropanol, propylene glycol, glycerin, low molecular weight polyethylene glycol, ethylene glycol based ester alcohols, and combinations thereof.

7. The oral composition of claim 1, wherein the cosolvent comprises propylene glycol.

8. The oral composition of claim 1, wherein the oral composition comprises from about 10 to about 28 wt % of water.

9. The oral composition of claim 1, wherein the oral composition comprises from about 35 to about 60 wt % of the cosolvent.

10. The oral composition of claim 1, wherein the molecular weight of the acidic copolymer is from about 5,000 Da to about 500,000 Da.

11. The oral composition of claim 1, wherein the oral composition comprises from about 15 to about 35 wt % of the acidic copolymer.

12. The oral composition of claim 1, wherein the acidic copolymer is an acidic copolymer comprising acidic acrylate monomeric units, acidic methacrylate monomeric units, or a combination thereof.

13. The oral composition of claim 1, wherein the acidic copolymer is chosen from Eudragit S100, Eudragit L100, Eudragit L100-55, AC210 and combinations thereof.

14. The oral composition of claim 1, further comprising an active agent.

15. The oral composition of claim 14, wherein the active agent is a whitening agent.

16. The oral composition of claim 15, wherein the whitening agent is a peroxide whitening agent.

17. The oral composition of claim 16, wherein the peroxide whitening agent is chosen from hydrogen peroxide, peroxides of alkali and alkaline earth metals, organic peroxy compounds, peroxy acids, peroxyacetic acid, urea hydrogen peroxide adduct, pharmaceutically-acceptable salts thereof, and mixtures thereof.

18. A method of delivering an oral composition to a dental structure comprising:
providing the oral composition of claim 1;
applying the oral composition to the dental structure; and
contacting the oral composition with an aqueous solution, thereby forming a polymeric film on the dental structure.
